(19) United States
(12) Patent Application Publication
(10) Pub. No.: US 2008/0233597 A1
(43) Pub. Date: Sep. 25, 2008

(54) Method of Detecting or Differentiating Rheumatoid Arthritis and Method of Determining Stage of Disease or Degree of Dysfunction with Regard to Rheumatoid Arthritis

(76) Inventors: Yasuhiko Shiina, Ibaraki (JP); Hiroshi Oda, Ibaraki (JP); Kosuke Seiki, Ibaraki (JP); Toshio Ushiyama, Shiga (JP); Yoshihiro Urade, Kyoto (JP); Naomi Eguchi, Osaka (JP); Yutaka Eguchi, Shiga (JP)

Correspondence Address: GREENBLUM & BERNSTEIN, P.L.C., 1950 ROLAND CLARKE PLACE, RESTON, VA, 20191, US

(21) Appl. No.: 10/573,324

(22) PCT Filed: Sep. 24, 2004
(86) PCT No.: PCT/JP04/14457

§ 371 Date: Mar. 24, 2006

(30) Foreign Application Priority Data
Sep. 26, 2003 (JP) 2003-336438

(51) Int. Cl. G01N 33/53; G01N 33/00; C07K 16/18; G01N 33/536

(52) U.S. Cl. 435/7.8; 436/86; 436/536; 530/387.1

(57) ABSTRACT

A method of readily detecting or differentiating rheumatoid arthritis, which has so far been diagnosed in a comprehensive manner based on various tests and clinical symptoms, and a method of readily and objectively estimating the stage of disease and the degree of dysfunction with regard to rheumatoid arthritis are provided.

The detection and differentiation of rheumatoid arthritis are performed by measuring the levels of L-PGDS in a sample such as a body fluid such that the measurement value is used as an index. Further, the stage of disease or the degree of dysfunction of a rheumatoid arthritis patient is determined using the measurement value as an index.

### TECHNICAL FIELD

The present invention relates to a method of detecting or differentiating rheumatoid arthritis. Specifically, the present invention relates to a method of readily detecting or differentiating rheumatoid arthritis by measuring human lipocalin-type prostaglandin D synthase in a sample such as a body fluid collected from a subject and also relates to a method of determining the stage of disease or the degree of dysfunction with regard to rheumatoid arthritis. Thus, the method of the present invention, wherein the stage of progression or the degree of severity of the disease is readily and objectively estimated, is useful for the clinical control of rheumatoid arthritis.

### BACKGROUND ART

Rheumatoid arthritis is characterized in chronic polyarthritis. The disease is a chronic nonspecific inflammatory disease of unknown cause, which is accompanied by a variety of extra-articular symptoms such as general fatigue, fever, and subcutaneous nodules. It has been reported that approximately 700,000 patients are affected with rheumatoid arthritis in Japan. The ratio of male to female patients is 1:4; the majority of the patients are females. The disease commonly develops in females in their 30s to 50s. In affected joints, destruction and deformities occur as well as swelling and pain over the course of the disease. As the disease progresses, patients become physically disabled as a result of dysfunction. In extreme cases, the patients become “bedridden.” Since rheumatoid arthritis is a disease of unknown cause, there is no certain treatment for it. However, some effective treatment methods have been developed for clinical applications. The most important points in these treatments for rheumatoid arthritis are to make an early and certain diagnosis so as to start treatment, and to understand the stage of progression and the degree of severity of the disease so as to select an adequate treatment method.

No specific symptoms or laboratory findings are available for diagnosing rheumatoid arthritis. Thus, detection or differentiation of the disease is carried out based on criteria in which relatively characteristic symptoms and findings are combined. In the past, criteria established by the American Rheumatism Association (ARA) were used. Since the revised ARA criteria were reported in 1987, clinical detection or differentiation has recently been carried out in accordance with the revised criteria (Table 1). The revised criteria consist of 7 criteria involving clinical symptoms and test methods. A patient shall be diagnosed as having rheumatoid arthritis if he or she has satisfied at least 4 of these 7 criteria. In addition, classification of the stage of disease (Stage) has been conducted based on clinical findings and radiographic images of affected joints. Clinical control is carried out by classifying a patient as corresponding to one of the four stages listed in Table 2. Further, classification of the degree of dysfunction (Class) has been conducted based on estimation of activities of daily living listed in Table 3. Also, clinical control is carried out based on this classification. (The aforementioned descriptions are cited from “The Basic Text of Rheumatism,” edited by the Kyoiku Kensyu Iinkai (Education and Training Committee) of the Japan Rheumatism Foundation, First edition, published in July 2002.)

Table 1

TABLE 1
Criteria for the Classification of Rheumatoid Arthritis

| | |
|---|---|
| 1) | Morning stiffness lasting at least 1 hour |
| 2) | Swelling of 3 or more joint areas |
| 3) | Swelling of hand joints (wrist, metacarpophalangeal, or proximal |
| | interphalangeal joints) |
| 4) | Symmetric swelling of joints |
| 5) | Abnormal findings on radiographic images of hand joints |
| 6) | Subcutaneous nodules |
| 7) | The presence of rheumatoid factor (blood test positive) |

Note that Criteria 1) through 4) must have been present for at least 6 weeks.

Table 2

TABLE 2
Classification of Stage of Disease
with regard to Rheumatoid Arthritis

Stage 1:
1. No radiographic evidence of bone destruction.
2. Osteoporosis may be present.
Stage 2:
1. Osteoporosis, with or without slight subchondral bone
destruction; slight cartilage destruction may be present.
2. Adjacent muscle atrophy.
Stage 3:
1. Cartilage and bone destruction in addition to osteoporosis.
2. Joint deformity such as subluxation, ulnar deviation,
or hyperextension without fibrous or bony ankylosis.
3. Extensive muscle atrophy.
Stage 4:
1. Fibrous or bony ankylosis.

Table 3

TABLE 3
Classification of Degree of Dysfunction
with regard to Rheumatoid Arthritis

| | |
|---|---|
| Class 1: | Complete functional capacity with ability to carry |
| | out all usual duties without handicaps |
| Class 2: | Functional capacity adequate for conducting normal |
| | activities despite discomfort or limited mobility |
| | of one or more joints |
| Class 3: | Functional capacity adequate for performing only few |
| | of the duties of usual occupation or of self-care |
| Class 4: | Largely or wholly incapacitated with patient bedridden |
| | or confined to wheelchair, permitting little or no |
| | self-care |

As described above, detection or differentiation of rheumatoid arthritis is carried out based on criteria involving a plurality of clinical symptoms and test methods in a comprehensive manner. It has been desired that a method of readily and objectively detecting or differentiating rheumatoid arthritis be established. In addition, the stage of disease and the degree of dysfunction with regard to rheumatoid arthritis are determined based on estimation of radiographic images of affected joints and of activities of daily living. Thus, determination of rheumatoid arthritis often differs depending on the medical institutions involved where a patient is examined. Therefore, indices whereby rheumatoid arthritis can readily and objectively be estimated have been awaited.

Meanwhile, lipocalin-type prostaglandin D synthase (hereafter abbreviated as L-PGDS) is an enzyme that catalyzes isomerization from PGH₂, which is a precursor common in a variety of prostaglandins, to PGD₂. In addition, L-PGDS also transports small hydrophobic molecules. Therefore, L-PGDS is known as a bifunctional protein that have the property of both an enzyme and a transporter (Urade Y. et al., Prostaglandin D synthase: Structure and function, Vitam Horm 2000; 58: 89-120). It has been reported that L-PGDS is detected in the blood of an advanced renal disease patient at a high concentration (Hoffmann A. et al., Molecular characterization of β-trace protein in human serum and urine: a potential diagnostic marker for renal diseases, Glycobiology 1997; 7: 499-506). Further, inventors of the present invention have elucidated that the L-PGDS concentration in a body fluid increases in the case of a patient with an early renal disease before the renal disease progresses (Hamano K. et al., Blood sugar control reverses the increase in urinary excretion of prostaglandin D synthase in diabetic patient, Nephron 2002; 92: 77-85). Furthermore, the inventors of the present invention have elucidated that the L-PGDS concentration in a body fluid increases as a result of the production of L-PGDS in arteriosclerotic plaque in the case of a patient with an ischemic heart disease (Eguchi Y. et al., Expression of lipocalin-type prostaglandin D synthase (β-trace) in human heart and its accumulation in the coronary circulation of angina patients, Proc Natl Acad Sci USA 1997; 94: 14689-94). As described above, the relationship between L-PGDS and renal diseases or ischemic heart diseases has been elucidated, although the relationship between L-PGDS and rheumatoid arthritis has never been discussed.

Non-Patent Document 1: Vitam Horm 2000; 58: 89-120

Non-Patent Document 2: Glycobiology 1997; 7: 499-506

Non-Patent Document 3: Nephron 2002; 92: 77-85

Non-Patent Document 4: Proc Natl Acad Sci USA 1997; 94: 14689-94

### DISCLOSURE OF THE INVENTION

It is an objective of the present invention to provide a method of readily detecting or differentiating rheumatoid arthritis that has been diagnosed based on various tests and clinical symptoms in a comprehensive manner. Further, it is another objective of the present invention to provide a method of readily and objectively estimating the stage of disease and the degree of dysfunction with regard to rheumatoid arthritis.

As a result of intensive studies to solve the above problem, the inventors of the present invention have found that differentiation, detection, and diagnosis of rheumatoid arthritis can be carried out by measuring the levels of L-PGDS in a sample such as a body fluid and using the obtained measurement value as an index. Further, the inventors have found that the stage of disease or the degree of dysfunction of a rheumatoid arthritis patient can be determined based on the measurement value as an index. This has led to the completion of the present study.

That is, the present invention relates to a method of detecting or differentiating rheumatoid arthritis, wherein the levels of L-PGDS in a sample such as a body fluid collected from a subject is measured.

In addition, the present invention relates to a method of determining the stage of disease or the degree of dysfunction with regard to rheumatoid arthritis, wherein the stage of disease or the degree of dysfunction is estimated based on a measurement value obtained by measuring the levels of L-PGDS in a sample such as a body fluid collected from a subject.

Specifically, the present invention is described in the following [1] to [14]:

[1] A method of detecting or differentiating rheumatoid arthritis, wherein the levels of human L-PGDS in a sample such as a body fluid collected from a subject is measured;
[2] The method of detecting or differentiating rheumatoid arthritis described in [1] above, wherein the levels of human L-PGDS in a sample such as a body fluid collected from a subject is measured, and the measurement value is compared with a cut-off value that has been predetermined based on measurement values of human L-PGDS in samples such as body fluids collected from healthy volunteers and/or patients with joint diseases other than rheumatoid arthritis;
[3] A method of determining the stage of disease with regard to rheumatoid arthritis, wherein the levels of human L-PGDS in a sample such as a body fluid collected from a subject is measured and the stage of disease with regard to rheumatoid arthritis is estimated based on the measurement value;
[4] The method of determining the stage of disease with regard to rheumatoid arthritis described in [3] above, wherein the levels of human L-PGDS in a sample such as a body fluid collected from a subject is measured and the measurement value is compared with a cut-off value that has been predetermined based on classification of measurement values of human L-PGDS in samples such as body fluids collected from rheumatoid arthritis patients in accordance with the stage of disease;
[5] A method of determining the degree of dysfunction with regard to rheumatoid arthritis, wherein the levels of human L-PGDS in a sample such as a body fluid is measured and the degree of dysfunction (severity) with regard to rheumatoid arthritis is estimated based on the measurement value;
[6] The method of determining the degree of dysfunction with regard to rheumatoid arthritis described in [5] above, wherein the levels of human L-PGDS in a sample such as a body fluid is measured and the measurement value is compared with the cut-off value that has been predetermined based on classification of measurement values of human L-PGDS in samples such as body fluids collected from rheumatoid arthritis patients in accordance with the degree of dysfunction (severity);
[7] The method described in any one of [1] to [6] above, wherein the levels of human L-PGDS in a sample such as a body fluid is measured by immunoassay;
[8] The method described in any one of [1] to [6] above, wherein the sample such as a body fluid is blood;
[9] The method described in any one of [1] to [6] above, wherein the sample such as a body fluid is a joint fluid;
[10] The method described in any one of [1] to [6] above, wherein the sample such as a body fluid is urine;
[11] An antibody specifically recognizing human L-PGDS for detection or differentiation of rheumatoid arthritis and for determination of the stage of disease or the degree of dysfunction with regard to rheumatoid arthritis; and

the antibody specifically recognizing human L-PGDS for detection or differentiation of rheumatoid arthritis and determination of the stage of disease or the degree of dysfunction with regard to rheumatoid arthritis, in which the antibody is a monoclonal antibody (also described as an anti-human L-PGDS monoclonal antibody);

[12] An agent for detection or differentiation of rheumatoid arthritis and an agent for determination of the stage of disease or the degree of dysfunction with regard to rheumatoid arthritis, comprising an antibody specifically recognizing human L-PGDS as an active ingredient; and

the agent for detection or differentiation of rheumatoid arthritis and the agent for determination of the stage of disease or the degree of dysfunction with regard to rheumatoid arthritis, in which the antibody is a monoclonal antibody;

[13] A kit for detection or differentiation of rheumatoid arthritis, comprising an antibody specifically recognizing human L-PGDS; and
[14] A human L-PGDS detection kit for detection or differentiation of rheumatoid arthritis, which is selected from a group consisting of (1) to (4) listed below:

(1) A reagent comprising an enzyme-labeled monoclonal antibody specifically recognizing human L-PGDS and a substrate solution;

(2) A reagent comprising a monoclonal antibody specifically recognizing human L-PGDS, an enzyme-labeled said monoclonal antibody or an enzyme-labeled polyclonal antibody specifically recognizing human L-PGDS, and a substrate solution;

(3) A reagent comprising a biotinylated monoclonal antibody specifically recognizing human L-PGDS, an enzyme-labeled avidin or streptavidin, a substrate solution, and a monoclonal antibody specifically recognizing human L-PGDS; and

(4) A reagent comprising a biotinylated monoclonal antibody specifically recognizing human L-PGDS or a biotinylated polyclonal antibody specifically recognizing human L-PGDS, an enzyme-labeled avidin or streptavidin, and a substrate solution.

Hereafter, the present invention will be described in greater detail.

This specification includes part or all of the contents as disclosed in the specification and/or drawings of Japanese Patent Application No. 2003-336438, which is a priority document of the present application.

### BRIEF DESCRIPTION OF THE DRAWINGS

FIG. 1 shows L-PGDS concentrations in the blood of healthy volunteers, gout patients, patients with pauciarticular arthritis, osteoarthritis patients, patients with seronegative spinal arthritis, and rheumatoid arthritis patients. The L-PGDS concentrations in the blood of rheumatoid arthritis patients are higher than those of healthy volunteers and any other patient groups.

FIG. 2 shows L-PGDS concentrations in the blood of rheumatoid arthritis patients in each stage of disease (Stage). The L-PGDS concentrations in the blood of the rheumatoid arthritis patients tend to increase significantly as the disease progresses.

FIG. 3 shows L-PGDS concentrations in the blood of rheumatoid arthritis patients at each degree of dysfunction (Class). The L-PGDS concentrations in the blood of the rheumatoid arthritis patients tend to significantly increase as the degree of dysfunction increases.

### BEST MODE FOR CARRYING OUT THE INVENTION

In the present invention, a sample used for the measurement of L-PGDS is a body fluid collected from a subject. Specific examples thereof include blood (serum, plasma, etc.), urine (casual urine, collected urine, etc.), and a joint fluid. Preferably, a method of measuring L-PGDS in the above samples is a measuring method whereby L-PGDS concentrations are accurately reflected. Examples thereof include immunoassay, enzyme activity assay, and capillary electrophoresis. In clinical practice, however, from the viewpoint of the necessity to readily and simultaneously measure large amounts of samples, qualitative or quantitative techniques using L-PGDS-specific monoclonal or polyclonal antibodies can be used. Examples of such techniques include enzyme immunoassay, double antibody sandwich ELISA, radioimmunoassay, latex agglutination immunoassay, fluorescence immunoassay, Western blotting, and an immunohistochemical method. Preferably, immunoassays such as enzyme immunoassay, radioimmunoassay, latex agglutination immunoassay, and fluorescence immunoassay can be used. For instance, an L-PGDS detection kit may be used, which has been established by the inventors of the present invention for sandwich ELISA using monoclonal antibodies (WO97/16461).

Alternatively, as a sample used for the measurement of L-PGDS, a section of joint tissue that has been collected from a subject may be used. In such case, rheumatoid arthritis can be detected or differentiated by a method of measuring L-PGDS, wherein a section of joint tissue is stained with anti-human L-PGDS antibodies such that the stained area is determined.

In the present invention, an agent for detection or differentiation of rheumatoid arthritis and an agent for determination of the stage of disease or the degree of dysfunction contain antibodies that specifically recognize human L-PGDS. Such antibodies that specifically recognize human L-PGDS involve those that are enzyme-labeled or those labeled by biotinylation.

In addition, the present invention encompasses the use of antibodies that specifically recognize human L-PGDS for the production of an agent for detection or differentiation of rheumatoid arthritis and an agent for determination of the stage of disease or the degree of dysfunction with regard to rheumatoid arthritis.

A kit for the present invention comprises the component reagent described below:
(1) (i) enzyme-labeled monoclonal antibodies; and (ii) a substrate solution.

The kit for the present invention comprises the reagent described below when sandwich ELISA is selected in a modified example of the kit described above:
(2) (i) monoclonal antibodies; (ii) enzyme-labeled monoclonal or polyclonal antibodies; and (iii) a substrate solution.

The kit for the present invention comprises the reagent described below when a biotin-avidin method is selected in a modified example of the above kit:
(3) (i) biotinylated monoclonal antibodies; (ii) enzyme-labelled avidin or streptavidin; and (iii) a substrate solution.

The kit for the present invention comprises the reagent described below when sandwich ELISA and a biotin-avidin method are selected in a modified example of the above kit:
(4) (i) monoclonal antibodies; (ii) biotinylated monoclonal antibodies or polyclonal antibodies; (iii) enzyme-labeled avidin or streptavidin; and (iv) a substrate solution.

The aforementioned substrate solution is a solution that contains a substrate, which changes detectably due to and an enzyme reaction caused by an enzyme, which is used for labeling an antibody. Examples of such substrate solution include a buffer solution containing p-nitrophenyl phosphate, a buffer solution containing o-phenylenediamine, and a buffer solution containing 4-methylumbelliferyl-β-galactoside, when antibodies are labeled with alkaline phosphatase (AP), horseradish peroxidase (HRPO), and β-galactosidase, respectively.

Preferably, two types of antibodies used in double antibody sandwich ELISA of (2) above are two types of anti-human L-PGDS monoclonal antibodies, which recognize different epitopes. Of them, the antibodies of one type (first antibodies) are solid phase antibodies on a carrier such as a microtiter plate. Thus, such antibodies can be used to immobilize L-PGDS. The antibodies of the other type (second antibodies) may be antibodies that can bind to the immobilized L-PGDS. Such antibodies are preferably labeled with a detectable substance for the subsequent detection. Such detectable substance can be biotin. Biotin can be detected using a known method. Preferably, the method comprises binding a streptavidin-peroxidase conjugate to biotin. The peroxidase used can be horseradish peroxidase. In addition, to detect such peroxidase, a substance which changes a color due to peroxidase action is preferably used.

To detect L-PGDS using the substance described above, at first, L-PGDS is allowed to bind to first antibodies, which are solid phase antibodies, on a carrier such as a microtiter plate. Next, biotin-labeled second antibodies are allowed to bind to the immobilized L-PGDS, and streptavidin-horseradish peroxidase conjugates are allowed to bind to the biotin portions. Lastly, a substance that changes a color due to horseradish peroxidase action is added thereto for color development such that quantitative assay of L-PGDS is carried out. When TM-Blue (Intergen) is used as a chromogenic substance, 0.5N sulfuric acid as a stop solution is added to the resultant, followed by agitation. Then, absorbance at 450 nm is determined using a plate reader or the like. Thus, quantitative assay of L-PGDS can be carried out.

In the present invention, rheumatoid arthritis can be detected or differentiated using, as an index, a measurement value of L-PGDS concentration that is measured by the above method. Further, by estimating the stage of disease or the degree of dysfunction with regard to rheumatoid arthritis based on the measurement value, clinical control of rheumatoid arthritis can be carried out. In addition, in the present invention, the term “clinical control” indicates understanding of clinical conditions (the stage of progression or the degree of severity) and follow-up observation.

When rheumatoid arthritis is detected or differentiated by the method of the present invention or when clinical control can be carried out by the method of the present invention, rheumatoid arthritis involves: malignant rheumatoid arthritis with complications including pleuritis, endocarditis, myocarditis, and peripheral neuritis derived from angiitis; and juvenile rheumatoid arthritis that develops in childhood. In addition, such rheumatoid arthritis involves rheumatoid arthritis with complications including secondary amyloidosis and an autoimmune disease such as Sjögren's syndrome or Hashimoto's thyroiditis.

When detecting or differentiating rheumatoid arthritis according to the present invention, at first, a reference interval is predetermined in the case of healthy volunteers. such reference interval varies depending on the type of sample used, such as a body fluid. Thus, a reference interval corresponding to the type of sample measured, such as a body fluid of a subject, is predetermined. Such reference interval can be predetermined based on measurement values obtained by measuring L-PGDS concentrations in samples such as body fluids of several or more healthy volunteers. L-PGDS concentrations can be measured in accordance with the above method. A person skilled in the art can adequately set a reference interval based on measurement values. A reference interval is obtained by, for example, the following equation: mean value (of measurement values) ±σ×standard deviation (σ=0.5, 1, 2, 3, or 5). A method known among persons skilled in the art can be used as a method for comparing the thus predetermined reference interval with a measurement value of the L-PGDS concentration in a sample such as a body fluid of a subject. Preferably, a method of comparing a cut-off value determined based on the above reference interval with a measurement value is used. In such case, if a measurement value of a subject is higher than the cut-off value, it can be determined that the subject is highly likely to be affected with rheumatoid arthritis. For instance, as such cut-off value, the upper limit of the reference interval obtained by the following equation can be used: mean value +σ×standard deviation (σ=0.5, 1, 2, 3, or 5).

In addition, a cut-off value can be predetermined in a following manner. For instance, L-PGDS concentrations in samples such as body fluids collected from healthy volunteers and/or patients affected with joint diseases other than rheumatoid arthritis are measured. Then, the distributions of L-PGDS concentrations in the case of healthy volunteers and/or patients affected with joint diseases other than rheumatoid arthritis are obtained. Then, the distribution of L-PGDS concentrations in the case of rheumatoid arthritis patients is obtained. Thereafter, the adequate cut-off value for L-PGDS concentration is predetermined based on diagnostic accuracy in terms of sensitivity, specificity, and the like for detection or differentiation of rheumatoid arthritis.

Subsequently, the L-PGDS concentration in a sample such as a body fluid collected from a subject is measured and is compared with the cut-off value. Thus, if the L-PGDS concentration in the sample exceeds the cut-off value, it is possible to detect or determine that the subject is affected with rheumatoid arthritis.

Also, the cut-off value is predetermined in relationship to the stage of disease (progression) or the degree of dysfunction (severity). Then, measurement values are compared with such predetermined cut-off value so that the stage of progression or the degree of severity can be determined by objectively estimating the stage of progression or the degree of severity. Thus, clinical control can be carried out. In a method of predetermining a cut-off value in relationship to the stage of disease (progression) or degree of dysfunction (severity), at first, L-PGDS concentrations in samples such as body fluids collected from a plurality of patients in each stage of disease or in each degree of dysfunction are measured. Then, based on the obtained measurement values, a reference interval for patients in each stage of disease or in each degree of dysfunction is predetermined. The reference interval varies depending on the type of sample used, such as a body fluid. Thus, a reference interval corresponding to the type of sample measured, such as a body fluid of a subject, is predetermined. A reference interval can be predetermined by, for example, the following equation: a mean value (of measurement values) ±σ×standard deviation (σ=0.5, 1, 2, 3, or 5), or using a percentile (of measurement values) between 5 and 95, between 10 and 90, or between 15 and 85. Next, based on the reference interval obtained above, the cut-off value is predetermined. To predetermine such cut-off value, the upper limit of the reference interval obtained by the following equation can be used: a mean value +σ×standard deviation (σ=0.5, 1, 2, 3, or 5), or using the 85^{th}, 90^{th}, or 95^{th} percentile.

In addition, by measuring L-PGDS concentrations in samples such as body fluids of rheumatoid arthritis patients in each stage of disease or in each degree of dysfunction, the distribution of L-PGDS concentrations in rheumatoid arthritis patients in each stage of disease or in each degree of dysfunction is obtained. Thus, based on diagnostic accuracy in terms of sensitivity or specificity upon determination of the stage of disease or the degree of dysfunction of rheumatoid arthritis patients, an adequate cut-off value of L-PGDS can be predetermined.

Preferably, the number of subjects that is necessary to predetermine a cut-off value as described above is 5 or more cases, and more preferably 10 or more cases; however, it is not limited thereto.

The present invention will be hereafter described in greater detail with reference to the following examples, although the technical scope of the present invention is not limited thereto.

### REFERENCE EXAMPLE

### Method of Measuring L-PGDS Concentrations in Body Fluids

L-PGDS concentrations in body fluids were measured by sandwich ELISA as described below.

First, anti-human L-PGDS monoclonal antibodies (clone: 7F5) capable of binding to human L-PGDS were diluted with 50 mM carbonate buffer solution (pH 9.6) to a concentration of 4.4 μg/ml. The resulting solution was added to wells of a 96-well microtiter plate in amounts of 300 μL/well. The plate was incubated overnight at 4° C. so that the solid-phase antibodies were obtained. Thereafter, the plate was washed three times with phosphate buffered saline (pH 7.4; hereafter abbreviated as PBS). Then, 0.2% casein-containing PBS (pH 7.4; hereafter referred to as a blocking solution) was added to wells of the plate in amounts of 300 μL/well and the plate was incubated at 30° C. for 90 minutes. Thus, blocking was carried out. Next, the plate subjected to blocking was washed three times with 0.05% Tween20-containing PBS (T-PBS). Then, an antigen solution (a standard solution or a sample body fluid that was diluted with the blocking solution) was added to wells of the plate in amounts of 100 μl/well and the plate was incubated at 30° C. for 90 minutes. After reaction took place, the plate was washed three times with T-PBS. Horseradish peroxidase-labelled anti-human L-PGDS monoclonal antibodies (clone: 1B7), which were diluted with a blocking solution to a concentration of 0.5 μg/ml, were added to wells of the plate in amounts of 100 μl/well and the plate was incubated at 30° C. for 90 minutes. After reaction took place, the plate was washed three times with T-PBS. Then, a chromogenic solution (ABTS solution; Boehringer Mannheim) was added to wells of the plate in amounts of 100 μl/well and the plate was incubated for 30 minutes at 30° C. After reaction took place, a stop solution (1.5% oxalic acid) was added to the plate in amounts of 100 μl/well, followed by agitation with a plate mixer. Thus, the reaction was terminated. Then, absorbance at 405 nm was determined using a commercially available plate reader.

The monoclonal antibodies (clones: 1B7 and 7F5) used in sandwich ELISA described above were prepared in a manner such that: pristane was intraperitoneally administered to mice in amounts of 1.0 ml; monoclonal-antibody-1B7-producing cells (1×10⁸ cells) and monoclonal-antibody-7F5-producing cells (1×10⁸ cells) were intraperitoneally implanted into different mice two weeks after the administration; and ascites of each mouse was collected two weeks after the implantation so as to be subjected to protein A affinity column chromatography. In addition, each cell line that produces one of the aforementioned monoclonal antibodies has a name corresponding to the name of the relevant monoclonal antibody. Both cell lines were deposited with the National Institute of Advanced Industrial Science and Technology, International Patent Organism Depositary (Central 6, 1-1-1 Higashi, Tsukuba, Ibaraki, Japan) under accession nos. FERM BP-5709 (date of the original deposit: Sep. 21, 1995) and FERM BP-5711 (date of the original deposit: Jun. 6, 1996), which correspond to 1B7 and 7F5, respectively.

In addition to the above cell lines, cell lines that produce anti-human L-PGDS monoclonal antibodies capable of binding to human L-PGDS have been deposited under accession nos. FERM BP-5710 (date of the original deposit: Sep. 21, 1995), FERM BP-5712 (date of the original deposit: Jun. 6, 1996), and FERM BP-5713 (date of the original deposit: Jun. 6, 1996), which correspond to clones 6F5, 9A6, and 10A3, respectively.

### Example 1

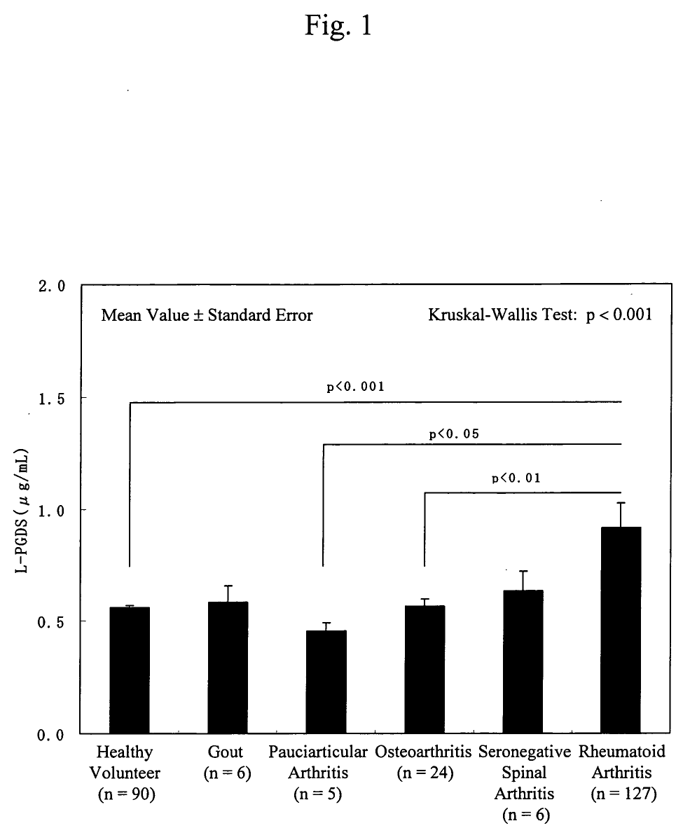

L-PGDS concentrations in the blood of healthy volunteers, patients with various types of arthritis (gout, pauciarticular arthritis, osteoarthritis, and seronegative spinal arthritis), and rheumatoid arthritis patients were measured. The results are shown in FIG. 1. L-PGDS concentrations in the blood of rheumatoid arthritis patients (n=127; 0.92±0.09 μg/ml (mean value ±standard error)) were significantly higher than those of healthy volunteers (n=90; 0.56±0.01 μg/ml) (p<0.001). Also, L-PGDS concentrations in the blood of rheumatoid arthritis patients were significantly higher than those of patients with pauciarticular arthritis (n=5; 0.46±0.04 μg/ml) and those of osteoarthritis patients (n=24; 0.56±0.04 μg/ml) (p<0.05 and p<0.01, respectively). In addition, the L-PGDS concentrations in the blood of rheumatoid arthritis patients tended to be higher than those of gout patients (n=6; 0.58±0.08 μg/ml) and those of patients with seronegative spinal arthritis (n=6; 0.64±0.11 μg/ml). Thus, it was considered that a joint disease considered to be a possible rheumatoid arthritis case would be highly likely to be rheumatoid arthritis when accompanied by a high L-PGDS concentration in blood. Therefore the measurement of L-PGDS concentrations in blood would be useful for detection or differentiation of rheumatoid arthritis.

### Example 2

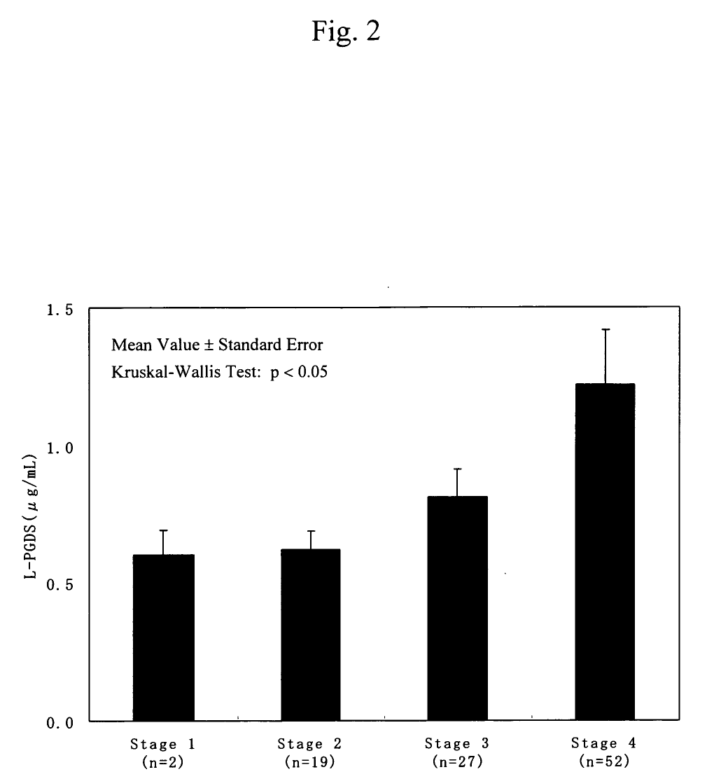

Based on clinical findings and radiographic images of affected joints, rheumatoid arthritis patients were classified into the four stages in accordance with the classification of the stage of disease with regard to rheumatoid arthritis established by the ARA. L-PGDS concentrations in the blood of the patients in each stage were determined. The results are shown in FIG. 2. It was found that the L-PGDS concentrations in blood tended to increase significantly in accordance with the advancement of the stage (p<0.05). Thus, it was considered that a rheumatoid arthritis patient with a high L-PGDS concentration in his or her blood would be highly likely to be in the advanced stage of disease. Therefore, the measurement of L-PGDS concentrations in blood would be useful for objective estimation of the stage of disease with regard to rheumatoid arthritis.

### Example 3

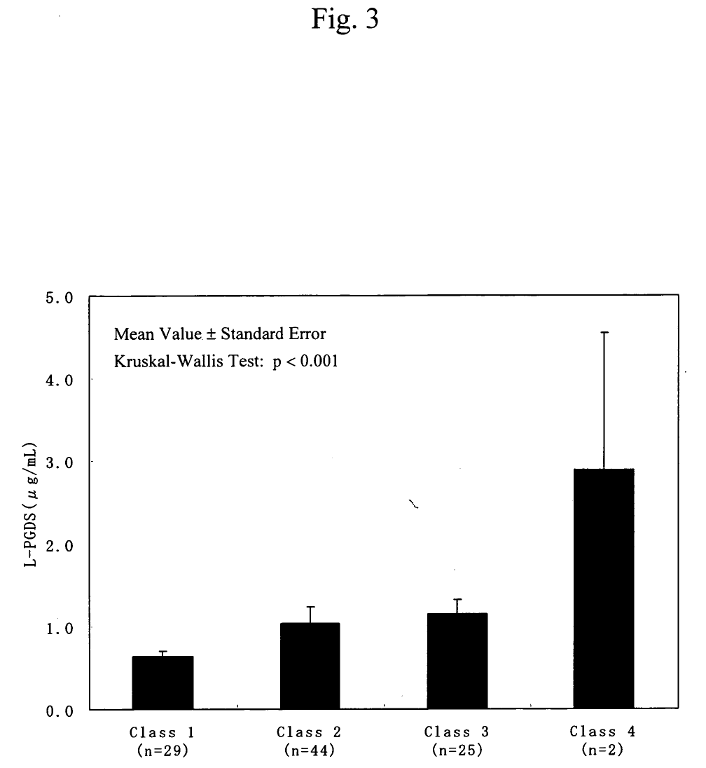

Based on estimation of activities of daily living, rheumatoid arthritis patients were classified into the four classes in accordance with the classification of the degree of dysfunction with regard to rheumatoid arthritis established by the ARA. L-PGDS concentrations in the blood of the patients in each class were measured. The results are shown in FIG. 3. It was found that the L-PGDS concentrations in blood tended to increase significantly in accordance with the advancement of the class (p<0.001). Thus, it was considered that a rheumatoid arthritis patient with a high L-PGDS concentration in blood would be highly likely to be in the advanced class in terms of the degree of dysfunction. Therefore, the measurement of L-PGDS concentrations in blood would be useful for objective estimation of the degree of dysfunction with regard to rheumatoid arthritis (severity).

### Example 4

A group of rheumatoid arthritis patients and a group of patients with joint diseases other than rheumatoid arthritis were subjected to the measurement of L-PGDS concentrations in blood. The upper limit of the reference interval of the L-PGDS concentrations in the blood of healthy volunteers (90 subjects) shown in Example 1 (mean value +2×standard deviation: 0.56+2×0.09=0.72 μg/ml) was determined to be the provisional cut-off value. Subjects of both groups were classified into the following two groups (resulting in four groups in total): a group with concentrations lower than or at the cut-off value (L-PGDS (−)); and a group with concentrations higher than the cut-off value (L-PGDS (+)). The results are listed in table 4. Based on the table, sensitivity, specificity, and diagnostic efficiency were calculated in terms of detection or differentiation of rheumatoid arthritis by measuring L-PGDS concentrations in blood, resulting in the following percentages: sensitivity: 50.4% (59/117); specificity: 88.1% (37/42); and diagnostic efficiency: 60.4% (96/159). Thus, it was considered that a joint disease patient considered to be a possible rheumatoid arthritis case would be highly likely to be affected with rheumatoid arthritis when the L-PGDS concentration in the blood collected from the patient is higher than the predetermined cut-off value. Therefore, the measurement of L-PGDS concentrations in blood would be useful for detection or differentiation of rheumatoid arthritis.

Table 4

TABLE 4
Results of Differential Diagnosis of Rheumatoid Arthritis
obtained by measuring L-PGDS concentrations in blood

| | | Group of Joint | |
|---|---|---|---|
| | Rheumatoid | Diseases other than | |
| | Arthritis Group | Rheumatoid Arthritis | |
| L-PGDS | 59 subjects | 5 | subjects |
| (+) | | | |
| L-PGDS | 58 subjects | 37 | subjects |
| (−) | | | |

### INDUSTRIAL APPLICABILITY

According to the present invention, a method of easily detecting or differentiating rheumatoid arthritis that has been diagnosed in a comprehensive manner based on various tests and clinical symptoms is provided. In addition, with the method of the present invention, the stage of disease (progression) and the degree of dysfunction (severity) can easily and objectively be estimated. Thus, the method of the present invention is extremely useful for detection or differentiation of rheumatoid arthritis and determination of the stage of disease and the degree of dysfunction of a patient.

All publications, patents, and patent applications cited herein are incorporated herein by reference in their entirety.

1. A method of detecting or differentiating rheumatoid arthritis, wherein the levels of human L-PGDS in a sample collected from a subject is measured.

2. The method of detecting or differentiating rheumatoid arthritis according to claim 1, wherein the levels of human L-PGDS in a sample collected from a subject is measured, and the measurement value is compared with a cut-off value that has been predetermined based on measurement values of human L-PGDS in samples collected from healthy volunteers and/or patients with joint diseases other than rheumatoid arthritis.

3. A method of determining the stage of disease with regard to rheumatoid arthritis, wherein the levels of human L-PGDS in a sample collected from a subject is measured and the stage of disease with regard to rheumatoid arthritis is estimated based on the measurement value.

4. The method of determining the stage of disease with regard to rheumatoid arthritis according to claim 3, wherein the levels of human L-PGDS in a sample collected from a subject is measured and the measurement value is compared with a cut-off value that has been predetermined based on classification of measurement values of human L-PGDS in samples collected from rheumatoid arthritis patients in accordance with the stage of disease.

5. A method of determining the degree of dysfunction with regard to rheumatoid arthritis, wherein the levels of human L-PGDS in a sample is measured and the degree of dysfunction (severity) with regard to rheumatoid arthritis is estimated based on the measurement value.

6. The method of determining the degree of dysfunction with regard to rheumatoid arthritis according to claim 5, wherein the levels of human L-PGDS in a sample is measured and the measurement value is compared with the cut-off value that has been predetermined based on classification of measurement values of human L-PGDS in samples collected from rheumatoid arthritis patients in accordance with the degree of dysfunction (severity).

7. The method according to claim 1, wherein the levels of human L-PGDS in a sample is measured by immunoassay.

8. The method according to claim 1, wherein the sample is a body fluid.

9. The method according to claim 1, wherein the sample is a joint fluid.

10. The method according to claim 1, wherein the sample is urine or blood.

11. An antibody specifically recognizing human L-PGDS for detection or differentiation of rheumatoid arthritis and for determination of the stage of disease or the degree of dysfunction with regard to rheumatoid arthritis.

12. An agent for detection or differentiation of rheumatoid arthritis and an agent for determination of the stage of disease or the degree of dysfunction with regard to rheumatoid arthritis, comprising an antibody specifically recognizing human L-PGDS as an active ingredient.

13. A kit for detection or differentiation of rheumatoid arthritis, comprising an antibody specifically recognizing human L-PGDS.

14. A human L-PGDS detection kit for detection or differentiation of rheumatoid arthritis, which is selected from a group consisting of (1) to (4) listed below:
(1) A reagent comprising an enzyme-labeled monoclonal antibody specifically recognizing human L-PGDS and a substrate solution;
(2) A reagent comprising a monoclonal antibody specifically recognizing human L-PGDS, an enzyme-labeled said monoclonal antibody or an enzyme-labeled polyclonal antibody specifically recognizing human L-PGDS, and a substrate solution;
(3) A reagent comprising a biotinylated monoclonal antibody specifically recognizing human L-PGDS, an enzyme-labeled avidin or streptavidin, a substrate solution, and a monoclonal antibody specifically recognizing human L-PGDS; and
(4) A reagent comprising a biotinylated monoclonal antibody specifically recognizing human L-PGDS or a biotinylated polyclonal antibody specifically recognizing human L-PGDS, an enzyme-labeled avidin or streptavidin, and a substrate solution.
